(19) United States
(12) Patent Application Publication
(10) Pub. No.: US 2020/0301398 A1
(43) Pub. Date: Sep. 24, 2020

(54) INFORMATION PROCESSING DEVICE, INFORMATION PROCESSING METHOD, AND PROGRAM

(71) Applicant: SONY CORPORATION, TOKYO (JP)

(72) Inventors: Kiminobu NISHIMURA, KANAGAWA (JP); Yoshihiro WAKITA, TOKYO (JP)

(21) Appl. No.: 16/089,158

(22) PCT Filed: Jan. 24, 2017
(86) PCT No.: PCT/JP2017/002200

§ 371 Date: Sep. 27, 2018

(30) Foreign Application Priority Data
Apr. 5, 2016 (JP) 2016-076028

(51) Int. Cl. G05B 19/4155; A61B 5/16

(52) U.S. Cl. CPC G05B 19/4155; G05B 2219/36488; A61B 5/165

(57) ABSTRACT

There is provided an information processing device including: a control unit configured to acquire emotion information obtained from biological information of a user and output control information related to an operation of an operation unit corresponding to the biological information of the user according to behavior information associated with the emotion information. In addition, there is provided an information processing method including, by a processor: acquiring emotion information obtained from biological information of a user and outputting control information related to an operation of an operation unit corresponding to the biological information of the user according to behavior information associated with the emotion information. This makes it possible to collect data related to emotions of a user more efficiently and realize more highly precise emotion estimation.

### TECHNICAL FIELD

The present disclosure relates to an information processing device, an information processing method, and a program.

### BACKGROUND ART

In recent years, various schemes for estimating feelings of users have been proposed. In addition, of the foregoing schemes, there are schemes for estimating feelings of users associated with operations of devices. For example, Patent Literature 1 discloses a feeling estimation method of estimating a feeling of a user when the user browses content.

### CITATION LIST

### Patent Literature

Patent Literature 1: JP 2014-222397A

### DISCLOSURE OF INVENTION

### Technical Problem

In the feeling estimation method disclosed in Patent Literature 1, however, a user is required to manually input feelings for a plurality of pieces of test content in advance. Therefore, in the feeling estimation method disclosed in Patent Literature 1, a burden on a user is large and it is difficult to react to more situations.

Accordingly, the present disclosure proposes an information processing device, an information processing method, and a program capable of collecting data related to emotions of a user more efficiently and realizing more highly precise emotion estimation.

### Solution to Problem

According to the present disclosure, there is provided an information processing device including: a control unit configured to acquire emotion information obtained from biological information of a user and output control information related to an operation of an operation unit corresponding to the biological information of the user according to behavior information associated with the emotion information.

### Advantageous Effects of Invention

According to the present disclosure, as described above, it is possible to collect data related to emotions of a user more efficiently and realize more highly precise emotion estimation. Note that the effects described above are not necessarily limitative. With or in the place of the above effects, there may be achieved any one of the effects described in this specification or other effects that may be grasped from this specification.

### BRIEF DESCRIPTION OF DRAWINGS

FIG. 1A is an explanatory diagram illustrating generation of a biology-emotion DB according to a first embodiment of the present disclosure.

FIG. 1B is an explanatory diagram illustrating an overview of operation control based on the biology-emotion DB according to the embodiment.

FIG. 2 is a functional block diagram according to the embodiment.

FIG. 3 is a diagram illustrating examples of behavior information output according to sensor information according to the embodiment.

FIG. 4 is a diagram illustrating a configuration example of data stored in a biology-emotion DB 390 according to the embodiment.

FIG. 5 is a diagram illustrating a configuration example of data stored in a behavior-emotion DB 395 according to the embodiment.

FIG. 6 is a flowchart illustrating a flow related to generation of a body-emotion DB 390 according to the embodiment.

FIG. 7 is a flowchart illustrating a flow of operation control using the biology-emotion DB 390 according to the embodiment.

FIG. 8 is a conceptual diagram illustrating an overview according to a second embodiment of the present disclosure.

FIG. 9 is a conceptual diagram illustrating an overview according to a third embodiment of the present disclosure.

FIG. 10 is a conceptual diagram illustrating an overview according to a fourth embodiment of the present disclosure.

FIG. 11 is a conceptual diagram illustrating an overview according to a fifth embodiment of the present disclosure.

FIG. 12 is a conceptual diagram illustrating an overview according to a sixth embodiment of the present disclosure.

FIG. 13 is a conceptual diagram illustrating an overview according to a seventh embodiment of the present disclosure.

FIG. 14 is a conceptual diagram illustrating an overview according to an eighth embodiment of the present disclosure.

FIG. 15A is a conceptual diagram illustrating an overview according to a ninth embodiment of the present disclosure.

FIG. 15B is a diagram illustrating another control example of the operation device according to the embodiment.

FIG. 16 is a conceptual diagram illustrating an overview according to a tenth embodiment of the present disclosure.

FIG. 17 is a conceptual diagram illustrating an overview according to an eleventh embodiment of the present disclosure.

FIG. 18 is a diagram illustrating a hardware configuration according to the present disclosure.

### MODE(S) FOR CARRYING OUT THE INVENTION

Hereinafter, (a) preferred embodiment(s) of the present disclosure will be described in detail with reference to the appended drawings. Note that, in this specification and the appended drawings, structural elements that have substantially the same function and structure are denoted with the same reference numerals, and repeated explanation of these structural elements is omitted.

Note that the description will be made in the following order.

1. Overview of the present disclosure
1.1. Points of note in the present disclosure

### 2. First Embodiment

2.1. Generation of biology-emotion DB
2.2. Operation control using biology-emotion DB
2.3. Functional configuration according to embodiment
2.4. Flow related to generation of biology-emotion DB
2.5. Flow of operation control using biology-emotion DB
2.6. Modification examples of first embodiment

### 3. Second Embodiment

### 4. Third Embodiment

### 5. Fourth Embodiment

### 6. Fifth Embodiment

### 7. Sixth Embodiment

### 8. Seventh Embodiment

### 9. Eighth Embodiment

### 10. Ninth Embodiment

### 11. Tenth Embodiment

### 12. Eleventh Embodiment

13. Hardware configuration example

### 14. Conclusion

### 1. Overview of the Present Disclosure

### <<1.1. Points of Note in the Present Disclosure>>

In recent years, methods of estimating emotions of a user according to biological information acquired from the user have been proposed. In addition, technologies for supplying services based on estimated emotions of the user are known.

However, in order to realize highly precise emotion estimation, many instruction signals for associating acquired biological information with emotions of a user at the time of obtaining of the biological information are required. The instruction signals may be, for example, information input by the user. However, in this case, a burden on the user is large and it is difficult to collect a large amount of instruction signals.

On the other hand, the user has various emotions and performs various behaviors daily. At this time, the foregoing emotions and behaviors are closely related in many cases. For example, in a case in which a user performs imaging using an imaging device such as a camera, the user is estimated to have an interest in a subject.

The present disclosure has been devised focusing on daily behaviors associated with the foregoing emotions. That is, according to the information processing device, the information processing method, and the program according to the present disclosure, a database in which biological information is associated with emotion information of a user when the user performs daily behaviors can be generated by recognizing daily behaviors which can serve as a weak instruction of emotions in advance.

In addition, according to the information processing device, the information processing method, and the program according to the present disclosure, emotions of a user can be estimated and various services or functions in accordance with the emotions can be supplied to the user by retrieving the database according to biological information acquired from the user.

Hereinafter, in the following description of an embodiment of the present disclosure, advantageous effects achieved from features will be described while exemplifying the features of a configuration of the information processing device realizing the foregoing functions. Note that emotions in the present disclosure may be analyzed as including feelings. That is, the emotions in the present disclosure may include feelings such as delight, anger, sorrow, and pleasure in addition to biological reactions of a user. The emotions according to the embodiment may be various states related to the user estimated from biological information.

### 2. First Embodiment

### <<2.1. Generation of Biology-Emotion DB>>

First, generation of a biology-emotion DB and a system configuration example according to the embodiment will be described. In the information processing method according to the embodiment, it is possible to generate a biology-emotion DB in which emotion information associated with behaviors of a user is associated with biological information acquired when the user performs the behaviors. At this time, in the information processing method according to the embodiment, the foregoing biological information and emotion information can be associated by referring to the behavior-emotion DB registered in advance.

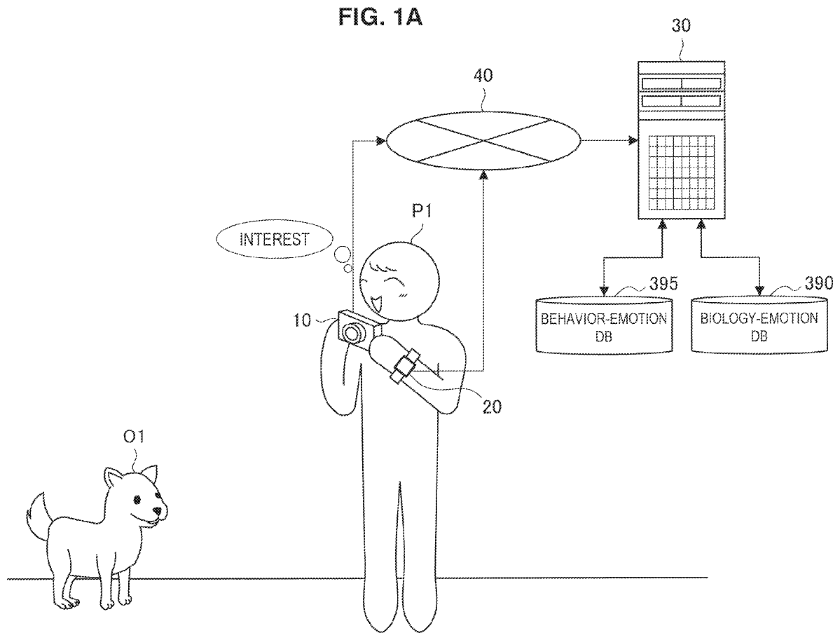

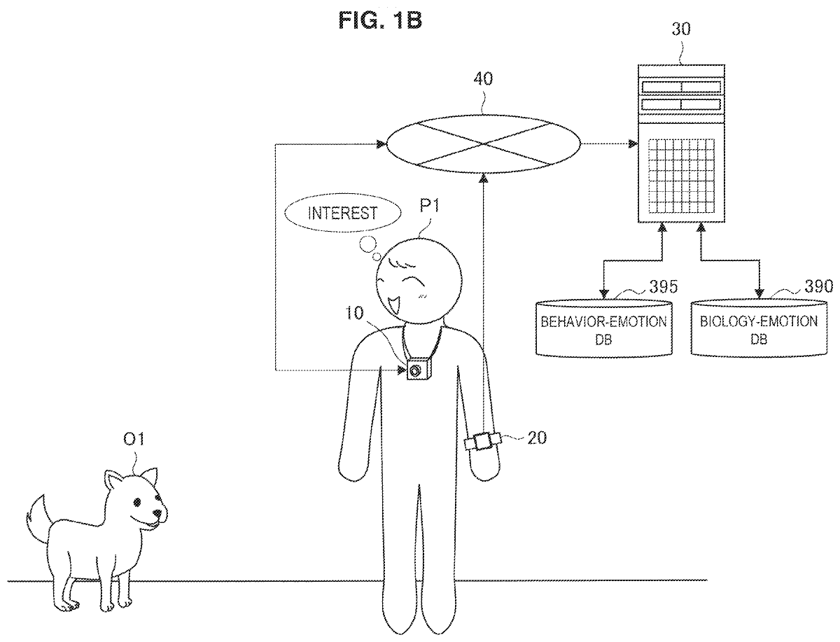

FIG. 1A is an explanatory diagram illustrating generation of a biology-emotion DB and a system configuration example according to the embodiment. Referring to FIG. 1A, an information processing system according to the embodiment includes an operation device 10, a wearable terminal 20, an information processing device 30, a biology-emotion DB 390, and a behavior-emotion DB 395. In addition, the operation device 10, the wearable terminal 20, and the information processing device 30 are connected via a network 40 so that mutual communication can be performed.

### (Operation Device 10)

In the generation of the biology-emotion DB 390 according to the embodiment, the operation device 10 may be any of various devices recognizing behaviors of a user P1. In an example illustrated in FIG. 1A, the operation device 10 may be any of various imaging devices, a smartphone that has an imaging function, or the like. The operation device 10 has a function of transmitting behavior information related to detected behaviors of the user P1 to the information processing device 30. For example, in the example illustrated in FIG. 1A, the operation device 10 detects that the user P1 photographs a subject O1 and transmits the detected information to the information processing device 30. More specifically, the operation device 10 may detect a behavior related to pressing of a shutter button by the user P1 and transmit behavior information regarding the behavior to the information processing device 30.

### (Wearable Terminal 20)

The wearable terminal 20 according to the embodiment may be any of various terminals acquiring biological information accompanied by a behavior of the user P1. In the example illustrated in FIG. 1A, the wearable terminal 20 acquires biological information of the user when the user photographs the subject O1. Therefore, the wearable terminal 20 may include various sensors acquiring biological information of the user P1. In addition, the wearable terminal 20 has a function of transmitting the acquired biological information of the user P1 to the information processing device 30.

### (Information Processing Device 30)

In the generation of the biology-emotion DB 390 according to the embodiment, the information processing device 30 has a function of generating the biology-emotion DB 390 by associating emotion information acquired from the behavior information of the user P1 of the operation device 10 with the biological information of the user P1. More specifically, the information processing device 30 can retrieve the behavior-emotion DB 395 registered in advance according to the behavior information received from the operation device 10 and acquire the emotion information associated with the behavior information of the user P1. In addition, the information processing device 30 can cause the biology-emotion DB 390 to be stored by associating the acquired emotion information with the biological information of the user P1 received from the wearable terminal 20. The information processing device 30 according to the embodiment may be any of various information processing terminals that have the foregoing functions. The information processing device 30 according to the embodiment may be, for example, a personal computer (PC), a smartphone, a tablet, or the like.

In the example illustrated in FIG. 1A, the information processing device 30 can receive behavior information related to the user P1 pressing the shutter button from the operation device 10 and acquire emotion information based on the behavior information from the behavior-emotion DB 395. At this time, in the foregoing behavior-emotion DB 395, for example, the pressing of the shutter button may be associated with emotion information related to “interest.” In this way, in the behavior-emotion DB 395 according to the embodiment, a behavior which the user P1 can perform and an estimated emotion of the user P1 when the user P1 performs the behavior may be stored. In the example illustrated in FIG. 1A, in a case in which the user P1 takes a picture, the user P1 is estimated to have “interest” in the subject O1. Therefore, the pressing of the shutter button and the emotion information related to “interest” are stored in the behavior-emotion DB 395 in association with each other. Note that the details of the behavior-emotion DB 395 according to the embodiment will be described later.

In addition, at this time, the information processing device 30 can cause the emotion information related to “interest” acquired from the behavior-emotion DB 395 and biological information received from the wearable terminal 20 to be stored in the biology-emotion DB 390 in association with each other. That is, the information processing device 30 can generate the biology-emotion DB 390 by associating the emotion information obtained from the behavior information of the user P1 photographing the subject O1 with the biological information of the user P1 acquired at the time of photographing.

### (Network 40)

The network 40 has a function of connecting the operation device 10, the wearable terminal 20, and the information processing device 30 to each other. The network 40 may include a public line network such as the Internet, a telephone line network, or a satellite communication network, or various local area networks (LANs), wide area networks (WANs), or the like including Ethernet (registered trademark). In addition, the network 40 may include a dedicated line network such as Internet Protocol-Virtual Private Network (IP-VPN). In addition, the network 40 may include a wireless communication network such as Wi-Fi (registered trademark) or Bluetooth (registered trademark).

The overview of the generation of the biology-emotion DB 390 according to the embodiment has been described above. As described above, the information processing device 30 according to the embodiment can generate the biology-emotion DB 390 according to the acquired behavior information and biological information of the user. The information processing device 30 according to the embodiment can dynamically collect instructions related to the biological information and the emotion information from daily behaviors of the user.

Note that in the foregoing description referring to FIG. 1A, the case in which the information processing device 30 acquires the behavior information of the user from the operation device 10 and the biological information of the user from the wearable terminal 20 has been described as the example. However, the system configuration example according to the embodiment is not limited to this example. For example, the information processing device 30 according to the embodiment may have a function of detecting a behavior of the user or a function of acquiring biological information of the user. That is, the operation device 10, the wearable terminal 20, and the information processing device 30 according to the embodiment may be realized as a single device.

In this case, the information processing device 30 may be, for example, an imaging device that has a function of generating the biology-emotion DB 390. In addition, in this case, the information processing device 30 may include various sensors acquiring biological information of the user. The system configuration and the functional configuration of the information processing device 30 according to the embodiment can be flexibly changed.

In addition, in the foregoing description, the case in which the operation device 10, the wearable terminal 20, and the information processing device 30 are connected via the network 40 has been described as the example. However, the system configuration example according to the embodiment is not limited to this example. The operation device 10 and the wearable terminal 20 can store data collected in an offline state in any of various storages. In this case, the information processing device 30 can generate the biology-emotion DB 390 by matching the behavior information and the biological information read from the storage with information of a time stamp or the like.

### <<2.2. Operation Control Using Biology-Emotion DB>>

Next, an overview of operation control using the biology-emotion DB according to the embodiment will be described. The information processing device 30 according to the embodiment has a function of controlling an operation of the operation device 10 according to the acquired biological information of the user. More specifically, the information processing device 30 can retrieve the biology-emotion DB 390 using the acquired biological information of the user to obtain the emotion information associated with the biological information. In addition, the information processing device 30 can retrieve the behavior-emotion DB 395 using the acquired emotion information and cause the operation device 10 to perform an operation corresponding to the obtained behavior information.

FIG. 1B is an explanatory diagram illustrating an overview of operation control based on the biology-emotion DB according to the embodiment. The example illustrated in FIG. 1B illustrates a case in which the information processing device 30 controls an operation of the operation device 10 according to the biological information of the user received from the wearable terminal 20. Note that in the example illustrated in FIG. 1B, the operation device 10 may be, for example, an imaging device such as a wearable camera.

In the example illustrated in FIG. 1B, the wearable terminal 20 may acquire the biological information of the user P1 at the time of viewing of the subject O1 and transmit the biological information to the information processing device 30. At this time, the information processing device 30 can retrieve the biology-emotion information 390 using the received biological information to acquire the emotion information associated with the biological information. In addition, the information processing device 30 retrieves the behavior-emotion DB 395 using the emotion information acquired above to acquire behavior information based on the emotion information. Specifically, the information processing device 30 can acquire the emotion information related to “interest” associated with the received biological information from the biology-emotion information 390. In addition, the information processing device 30 can acquire the behavior information related to a “shutter pushing” operation associated with “interest” from the behavior-emotion DB 395.

In addition, the information processing device 30 can perform the operation control of the operation device 10 according to the behavior information acquired above. Specifically, the information processing device 30 may output the control information for pushing a shutter in the operation device 10. In this case, the operation device 10 can photograph the subject O1 by performing a shutter manipulation according to the received control information. That is, the information processing device 30 according to the embodiment can cause the operation device 10 to perform an operation associated with the emotion information estimated from the acquired biological information of the user. According to the foregoing function of the information processing device 30, by causing the operation device 10 to automatically perform an operation corresponding to an estimated emotion of the user, it is possible to considerably reduce a manipulation burden on the user and supply a high-value service to the user.

### <<2.3. Functional Configuration According to Embodiment>>

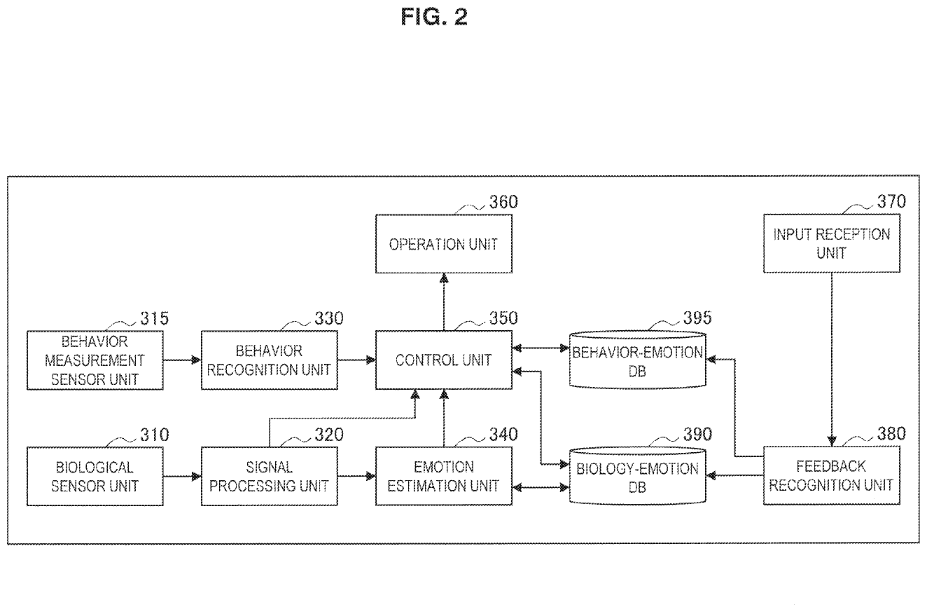

Next, a functional configuration of the information processing system according to the embodiment will be described. FIG. 2 is a functional block diagram according to the embodiment. Referring to FIG. 2, the information processing system according to the embodiment includes a biological sensor unit 310, a behavior measurement sensor unit 315, a signal processing unit 320, a behavior recognition unit 330, an emotion estimation unit 340, a control unit 350, an operation unit 360, an input reception unit 370, and a feedback recognition unit 380. In addition, the information processing system according to the embodiment may include the biology-emotion DB 390 and the behavior-emotion DB 395.

In addition, as described above, the system configuration according to the embodiment may be flexibly designed in accordance with a service to be supplied or a specification of each device. For example, the information processing device 30 according to the embodiment may include the signal processing unit 320, the behavior recognition unit 330, the emotion estimation unit 340, the control unit 350, and the feedback recognition unit 380. The operation device 10 according to the embodiment may include the behavior measurement sensor unit 315, the operation unit 360, and the input reception unit 370. In addition, the wearable terminal 20 according to the embodiment may include the biological sensor unit 310. Note that the wearable terminal 20 may further include the behavior measurement sensor unit 315. In this case, the wearable terminal 20 can collect behavior information in addition to the biological information of the user.

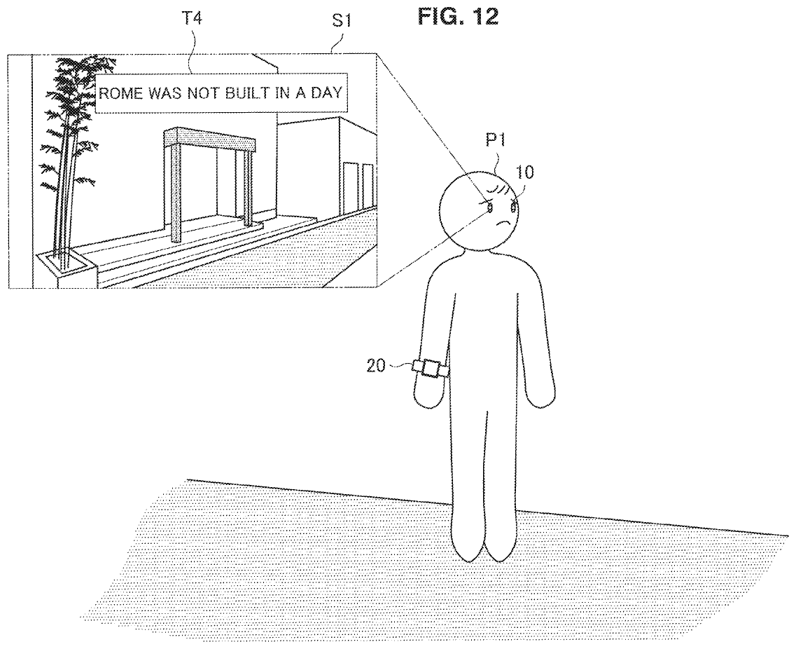

On the other hand, the information processing device 30 according to the embodiment may have the entire configuration illustrated in FIG. 2. In this case, the information processing device 30 can also function as the operation device 10 and the wearable terminal 20 illustrated in FIGS. 1A and 12B. Hereinafter, each configuration described above will be described in detail.

### (Biological Sensor Unit 310)

The biological sensor unit 310 has a function of measuring biological information of the user. Therefore, the biological sensor unit 310 may include various sensors measuring biological information of the user. The biological sensor unit 310 can include, for example, a sensor measuring pulse waves, perspiration, a blood pressure, myoelectricity, an electrocardiogram, an ocular potential, brain waves, a pupil aperture ratio, a body temperature, or the like of the user.

### (Behavior Measurement Sensor Unit 315)

The behavior measurement sensor unit 315 has a function of detecting sensor information related to a behavior of the user including a device manipulation. Therefore, the behavior measurement sensor unit 315 may include various sensors detecting sensor information related to a behavior of the user. The behavior measurement sensor unit 315 may detect sensor information related to a behavior of the user using, for example, one of an acceleration sensor, a gyro sensor, an atmospheric pressure sensor, a temperature sensor, a humidity sensor, a myoelectric sensor, a sound sensor, a pressure sensor, an imaging sensor, a microphone, a button, and a switch or a combination thereof. In addition, the behavior measurement sensor unit 315 may further include each sensor included in the biological sensor unit 310 described above.

### (Signal Processing Unit 320)

The signal processing unit 320 has a function of performing noise removal or feature extraction on some or all of the biological information acquired by the biological sensor unit 310. At this time, the signal processing unit 320 may perform both the noise removal and the feature extraction on the biological information acquired by the biological sensor unit 310 or may perform any one thereof. The signal processing unit 320 can perform a process in accordance with a kind of biological information acquired by the biological sensor unit 310.

### (Behavior Recognition Unit 330)

The behavior recognition unit 330 has a function of recognizing a behavior of the user according to the sensor information related to a behavior of the user acquired by the behavior measurement sensor unit 315 and outputting the behavior information. At this time, the behavior recognition unit 330 may recognize a behavior of the user according to a statistical scheme or a machine learning scheme such as a support vector machine (SVM), a neural network, or a regression model.

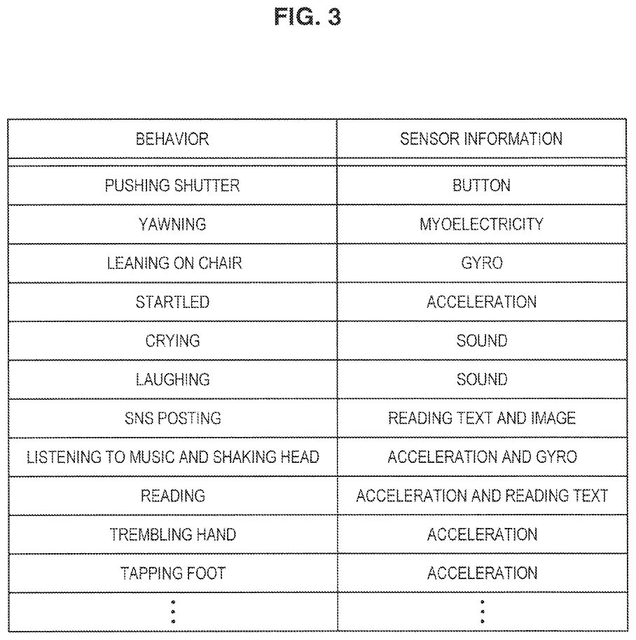

FIG. 3 is a diagram illustrating examples of behavior information output according to the sensor information by the behavior recognition unit 330. Referring to FIG. 3, the behavior recognition unit 330 can recognize a behavior of the user, such as pushing of the shutter, for example, according to a button manipulation detected by the behavior measurement sensor unit 315. In addition, the behavior recognition unit 330 may recognize a behavior of the user, such as yawning, according to myoelectric potential information detected by the behavior measurement sensor unit 315.

### (Emotion Estimation Unit 340)

The emotion estimation unit 340 has a function of outputting emotion information estimated from biological information with reference to the biology-emotion DB 390 according to the biological information output from the signal processing unit 320. At this time, the foregoing emotion information output by the emotion estimation unit 340 may include the kinds or degrees of emotions.

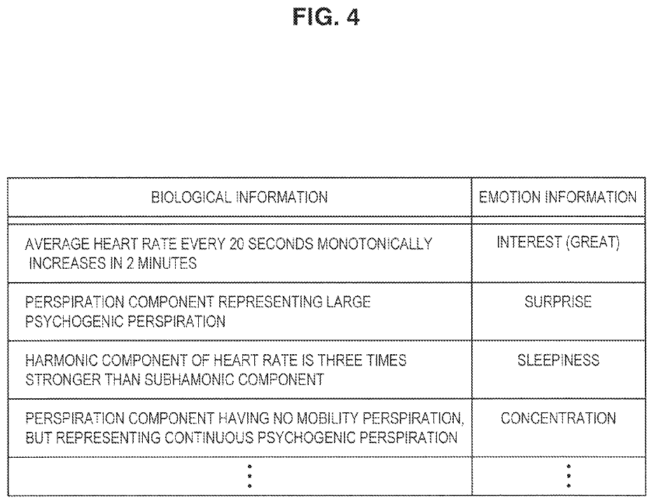

FIG. 4 is a diagram illustrating a configuration example of data stored in the biology-emotion DB 390 according to the embodiment. More specifically, FIG. 4 illustrates a configuration example of individualized data stored in the individualized biology-emotion DB 390. That is, in the biology-emotion DB 390, biological information and emotion information based on individual features are stored in association. The emotion estimation unit 340 can output the emotion information associated with the biological information by comparing features of the biological information output from the signal processing unit 320 to features of the biological information stored in the biology-emotion DB 390.

At this time, for example, the emotion estimation unit 340 may output emotion information in accordance with each user or each behavior of the user by processing the biological information output from the signal processing unit 320 and information stored in the biology-emotion DB 390 in accordance with a statistical scheme or a machine learning scheme such as SVM, a neural network, or a regression model.

### (Control Unit 350)

The control unit 350 has a function of acquiring emotion information obtained from biological information of the user and outputting control information related to an operation of the operation unit 360 corresponding to the biological information of the user according to behavior information associated with the emotion information. That is, the control unit 350 has a function of acquiring the behavior information associated with the emotion information according to the emotion information output by the emotion estimation unit 340 from the behavior-emotion DB 395. In addition, the control unit 350 has a function of controlling an operation of the operation unit 360 according to the behavior information acquired above.

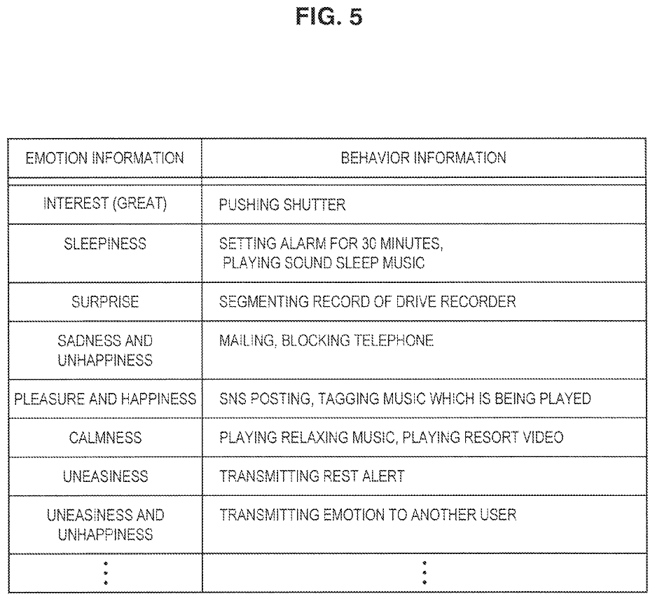

FIG. 5 is a diagram illustrating a configuration example of data stored in the behavior-emotion DB 395 according to the embodiment. Referring to FIG. 5, the behavior information and the emotion information are stored in association in the behavior-emotion DB 395. As illustrated in FIG. 5, the behavior information stored in the behavior-emotion DB 395 may include information indicating an operation that the operation unit 360 is caused to perform. The control unit 350 can acquire behavior information associated with the emotion information output by the emotion estimation unit 340 and control an operation that the operation unit 360 is caused to perform according to the behavior information.

In addition, the behavior information stored in the behavior-emotion DB 395 may include an operation related to a past device manipulation performed by the user. The control unit 350 can cause the operation unit 360 to perform an operation similar to the foregoing device manipulation according to the behavior information associated with the acquired kinds of emotion and feeling. For example, referring to FIG. 5, the behavior information stored in the behavior-emotion DB 395 includes information related to a device manipulation, such as pushing of the shutter, performed by the user in the past. At this time, the control unit 350 can cause the operation unit 360 to perform an operation of pushing the shutter according to the fact that the emotion information output by the emotion estimation unit 340 indicates great interest.

In addition, the behavior information stored in the behavior-emotion DB 395 may include information related to a device operation to be recommended to the user. The control unit 350 can cause the operation unit 360 to perform the device operation to be recommended to the user according to the behavior information based on the acquired kinds of emotion and feeling. For example, referring to FIG. 5, the behavior information stored in the behavior-emotion DB 395 includes information related to the device operation to be recommended to the user, such as transmission of a rest alert. At this time, the control unit 350 can cause the operation unit 360 to transmit a rest alert according to the fact that the emotion information output by the emotion estimation unit 340 indicates uneasiness.

Further, the control unit 350 can acquire the emotion information associated with the behavior information according to the acquisition of the behavior information of the user and cause the biology-emotion DB to store the emotion information and the acquired biological information in association with each other. That is, the control unit 350 can acquire the emotion information from the behavior-emotion information 395 according to the behavior information output by the behavior recognition unit 330. In addition, the control unit 350 can cause the biology-emotion DB 390 to store the emotion information acquired above and the biological information output by the signal processing unit 320 in association with each other.

### (Operation Unit 360)

The operation unit 360 has a function of performing various operations according to the control information output from the control unit 350. For example, the operation unit 360 may perform a shutter pushing operation according to the control information output from the control unit 350. The operation unit 360 can perform various operations in accordance with a specification of the device including the operation unit 360.

### (Input Reception Unit 370)

The input reception unit 370 has a function of receiving an input manipulation by the user. Therefore, the input reception unit 370 may include input devices such as a keyboard, a mouse, a touch panel, a microphone, and a button.

### (Feedback Recognition Unit 380)

The feedback recognition unit 380 has a function of recognizing feedback from the user in response to an operation of the operation unit 360. At this time, the feedback recognition unit 380 may recognize feedback from the user according to a user input manipulation received by the input reception unit 370. In addition, the feedback may include evaluation of the user in regard to an operation of the operation unit 360.

In addition, the feedback recognition unit 380 may control correction related to correlation strength between the corresponding biological information and emotion information according to the feedback. The feedback recognition unit 380 can perform control such that the correlation strength between the corresponding biological information and emotion information is weakened according to the fact that the evaluation of the user in regard to the operation of the operation unit 360 is negative. Alternatively, the feedback recognition unit 380 can perform control such that the correlation strength between the corresponding biological information and emotion information is strengthened according to positive evaluation of the user in regard to the operation of the operation unit 360.

For example, in a case in which the control unit 350 controls the operation unit 360 such that the shutter is pushed according to the acquired biological information, the feedback recognition unit 380 can recognize a manipulation by the user on a photographed image. At this time, for example, the feedback recognition unit 380 may perform control such that the correlation strength between the biological information and the emotion information used when the control unit 350 controls the operation unit 360 is weakened according to the fact that the user deletes a photographed image.

On the other hand, for example, the feedback recognition unit 380 can also perform control such that the correlation strength between the biological information and the emotion information used when the control unit 350 controls the operation unit 360 is strengthened according to the fact that the user reserves a photographed image. At this time, the feedback recognition unit 380 may strengthen, for example, a weight of a support vector in a model generated using SVM. In addition, for example, the feedback recognition unit 380 can also strengthen a connection weight in a constructed neural network model.

In the foregoing function of the feedback recognition unit 380 according to the embodiment, the correlation strength between the biological information and the emotion information can be appropriately corrected in accordance with feedback of the user. Thus, it is possible to realize more highly precise emotion estimation.

In addition, the feedback recognition unit 380 may control the correction related to the association between the corresponding behavior information and emotion information according to the feedback from the user. The feedback recognition unit 380 can also perform control such that the association between the corresponding behavior information and emotion information is deleted according to the fact that the evaluation of the user in regard to the operation of the operation unit 360 is negative.

For example, the feedback recognition unit 380 can also delete the association between the behavior information and the emotion information used when the control unit 350 controls the operation unit 360 according to the fact that the user deletes photographed images a predetermined number of time or more.

In the foregoing function of the feedback recognition unit 380 according to the embodiment, the association between the biological information and the emotion information can be appropriately corrected in accordance with feedback of the user. Thus, it is possible to realize supply of a higher-value service.

### (Biology-Emotion DB 390)

The biology-emotion DB 390 is an information database that stores the acquired biological information and emotion information of the user in association therewith. Here, the foregoing emotion information may be emotion information acquired from the behavior-emotion information 395 by the control unit 350 according to the behavior information output by the behavior recognition unit 330. As described with reference to FIG. 4, the biology-emotion DB 390 according to the embodiment can store the features of the biological information and the kinds and degrees of emotions in association with each other.

In addition, the biology-emotion DB 390 according to the embodiment may be an information database generated for each user or each behavior of the user. The control unit 350 can realize more highly precise emotion estimation in accordance with characteristics of the user by generating the biology-emotion DB 390 for each user or each behavior of the user.

### (Behavior-Emotion DB 395)

The behavior-emotion DB 395 is an information database that stores behavior information and emotion information of the user in association therewith. The behavior-emotion DB 395 according to the embodiment may be a database preset in accordance with the function of the operation unit 360. As described with reference to FIG. 5, the behavior-emotion DB 395 according to the embodiment can store the kinds of degrees of emotions and operations that the operation unit 360 is caused to perform in association with each other.

### <<2.4. Flow Related to Generation of Biology-Emotion DB>>

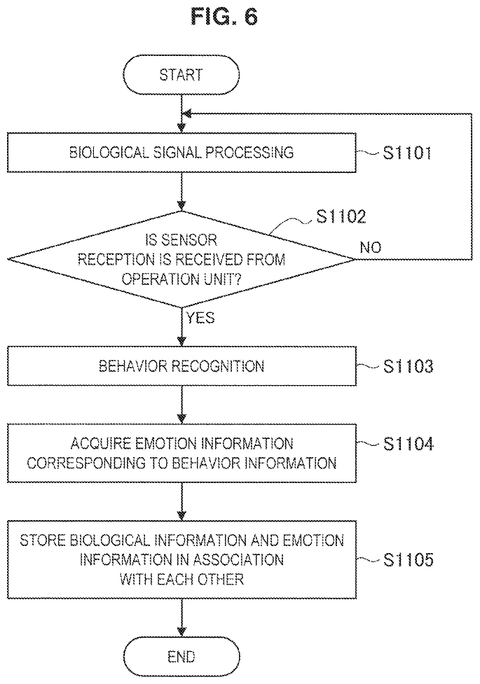

The functional configuration of the information processing system according to the embodiment has been described above. Next, a flow related to generation of the biology-emotion DB 390 according to the embodiment will be described in detail. FIG. 6 is a flowchart illustrating the flow related to the generation of the biology-emotion DB 390 according to the embodiment. Note that in the following description, a case in which the information processing device 30 includes the signal processing unit 320, the behavior recognition unit 330, the emotion estimation unit 340, the control unit 350, and the feedback recognition unit 380 will be described as an example. In addition, at this time, the operation device 10 may be a device that includes the behavior measurement sensor unit 315, the operation unit 360, and the input reception unit 370. In addition, the wearable terminal 20 may include the biological sensor unit 310.

Referring to FIG. 6, the signal processing unit 320 of the information processing device 30 first performs signal processing on the biological information acquired from the biological sensor unit 310 of the wearable terminal 20 (S1101). As described above, at this time, the signal processing unit 320 may perform noise removal in the acquired biological information, feature extraction in accordance with a kind of biological information, or the like.

More specifically, for example, in a case in which biological information acquired from the biological sensor unit 310 includes a pulse wave, the signal processing unit 320 can perform drift removal by a highpass filter and feature extraction by heartbeat interval calculation or the like. In addition, in a case in which the acquired biological information includes information regarding perspiration, the signal processing unit 320 can perform downsampling, reverse convolution calculation using an impulse response function, or the like. As described above, when the signal processing unit 320 performs appropriate signal processing in accordance with the biological information, it is possible to realize more efficient emotion estimation. In addition, the signal processing unit 320 may retain the biological information output in response to the foregoing process for a predetermined time.

Subsequently, the behavior recognition unit 330 of the information processing device 30 determines whether the sensor information related to a behavior of the user is received from the behavior measurement sensor unit 315 of the operation device 10 (S1102). Here, in a case in which the sensor information related to the behavior of the user may not be received from the behavior measurement sensor unit 315 (NO in S1102), the information processing device 30 may return to step S1101 to repeatedly perform signal processing related the biological information.

Conversely, in a case in which the sensor information related to the behavior of the user is received from the behavior measurement sensor unit 315 (YES in S1102), the behavior recognition unit 330 performs behavior recognition according to the received sensor information (S1103). At this time, for example, the behavior recognition unit 330 can recognize that the user takes a picture according to the fact that the sensor information related to the shutter button is received from the behavior measurement sensor unit 315.

In addition, for example, the behavior recognition unit 330 can also recognize that the user yawns by processing the sensor information acquired by the acceleration sensor included in the behavior measurement sensor unit 315 in accordance with a statistical scheme or a machine learning scheme such as SVM, a neural network, or a regression model.

In addition, for example, the behavior recognition unit 330 may recognize that the user laughs or cries by processing sound information acquired by a microphone included in the behavior measurement sensor unit 315 in accordance with the foregoing statistical scheme or machine learning scheme.

Subsequently, the control unit 350 of the information processing device 30 acquires the emotion information associated with the behavior information of the user recognized in step S1103 from the behavior-emotion DB 395 (S1104).

Subsequently, the control unit 350 causes the biological information subjected to the signal processing in step S1101 and the emotion information acquired in step S1104 to be stored in the biology-emotion DB 390 in association therewith (S1105).

The flow related to the generation of the biology-emotion DB 390 according to the embodiment has been described above. As described above, the information processing device 30 according to the embodiment can cause the emotion information associated with the behavior information of the user and the acquired biological information to be stored in the biology-emotion DB 390 in association with each other. In the foregoing function of the information processing device 30 according to the embodiment, the biology-emotion DB 390 can be generated for each user or each behavior of the user. Thus, it is possible to realize high precise emotion estimation in accordance with the characteristic of the user.

### <<2.5. Flow of Operation Control Using Biology-Emotion DB>>

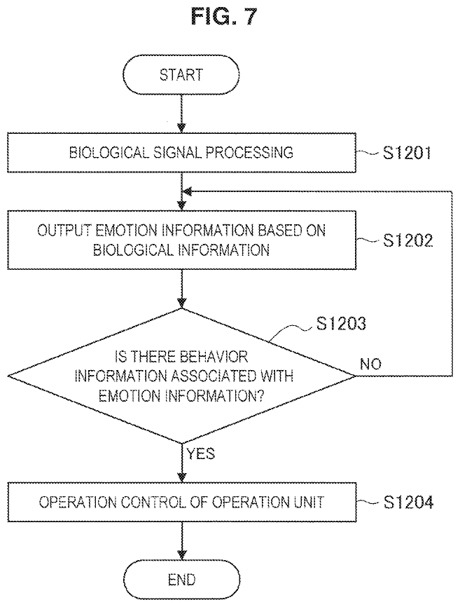

Next, a flow of operation control using the biology-emotion DB 390 according to the embodiment will be described in detail. FIG. 7 is a flowchart illustrating the flow of the operation control using the biology-emotion DB 390 according to the embodiment. Note that in the following description, a case in which the information processing device 30 includes the signal processing unit 320, the behavior recognition unit 330, the emotion estimation unit 340, the control unit 350, and the feedback recognition unit 380 will be described as an example. In addition, at this time, the operation device 10 may be a device that includes the behavior measurement sensor unit 315, the operation unit 360, and the input reception unit 370. In addition, the wearable terminal 20 may include the biological sensor unit 310.

Referring to FIG. 7, the signal processing unit 320 of the information processing device 30 first performs signal processing on the biological information acquired from the biological sensor unit 310 of the wearable terminal 20 (S1201). At this time, the signal processing unit 320 may perform a process similar to the process described in step S1101 of FIG. 6.

Subsequently, the emotion estimation unit 340 of the information processing device 30 outputs the emotion information estimated from the biological information with reference to the biology-emotion DB 390 according to the biological information processed in step S1201 (S1202). Note that, at this time, the output emotion information may include the kinds and degrees of emotions.

Subsequently, the control unit 350 of the information processing device 30 determines whether there is the behavior information associated with the emotion information with reference to the behavior-emotion DB 395 according to the emotion information output in step S1202 (S1203). That is, the control unit 350 may determine whether the kinds and degrees of emotions included in the emotion information match preset conditions.

Here, in a case in which there is no behavior information which matches the emotion information output in step S1202 (NO in S1203), the information processing device 30 returns to step S1202 to repeatedly perform the following processes.

Conversely, in a case in which the behavior information which matches the emotion information output in step S1202 is acquired (YES in S1203), the control unit 350 performs operation control of the operation unit 360 of the operation device 10 according to the acquired behavior information (S1204). That is, the control unit 350 may transmit the control information related to the operation of the operation unit 360.

The flow of the operation control using the biology-emotion DB 390 according to the embodiment has been described above. As described above, the information processing device 30 according to the embodiment can acquire the behavior information associated with the emotion information according to the emotion information estimated form the biological information of the user. In addition, the information processing device 30 can control the operation of the operation unit 360 according to the acquired behavior information. In the foregoing function of the information processing device 30 according to the embodiment, the operation unit 360 can be caused to perform various processes based on the estimated emotions of the user. Thus, it is possible to considerably reduce a processing burden on the user and supply a higher-value service.

### <<2.6. Modification Examples of First Embodiment>>

The functions of the information processing device 30 according to the embodiment have been described above in detail. As described above, the information processing device 30 according to the embodiment has the function of generating the biology-emotion DB 390 according to the behavior information and the biological information of the user. In addition, the information processing device 30 according to the embodiment has the function of controlling the operation of the operation unit 360 corresponding to the biological information of the user using the generated biology-emotion DB 390. On the other hand, the functions of the information processing device 30 described above can be appropriately modified in accordance with a specification, running, or the like of the information processing device 30. Hereinafter, modification examples of the embodiment will be described.

### (Generation of Biology-Behavior DB)

In the foregoing description, the case in which the information processing device 30 generates the biology-emotion DB 390 by associating the biological information of the user with the emotion information associated with the behavior information has been described as the example. On the other hand, the information processing device 30 according to the embodiment may associate the biological information with the behavior information without associating the emotion information. That is, the information processing device 30 can also generate a biology-behavior DB in which the acquired biological information and behavior information are associated.

In this case, even in a case in which the emotion information associated with the behavior information is not registered, the information processing device 30 can cause the operation unit 360 to perform an operation based on the biological information of the user. In addition, in a case in which the emotion information associated with the behavior information is registered later, the information processing device 30 can generate the biology-emotion DB 390 by converting the behavior information stored in the biology-behavior DB into the registered emotion information.

### (Utilization of Generated Biology-Emotion DB)

In addition, in the foregoing description, the case in which the information processing device 30 generates the biology-emotion DB 390 according to the behavior information and the biological information of the user has been described as the example. On the other hand, the information processing device 30 according to the embodiment can use the generated biology-emotion DB 390 to give an instruction of the biological information acquired by the different biological sensor unit 310. In this case, the information processing device 30 can correct the biology-emotion DB 390 in the instruction of the emotion information estimated according to the generated biology-emotion DB 390.

### (Individual Authentication Using Biology-Emotion DB)

In addition, in the foregoing description, the operation control of the operation unit 360 using the biology-emotion DB 390 by the information processing device 30 has been mainly described. However, the information processing device 30 according to the embodiment can also perform individual authentication using the biology-emotion DB 390. In this case, the information processing device 30 can determine feedback from the user according to the biology-emotion DB 390 generated for each user and perform individual authentication. The information processing device 30 may perform the foregoing individual authentication according to the fact that the feedback from the user in regard to an operation of the operation unit 360 matches information stored in the biology-emotion DB 390 to a predetermined extent or more.

(Utilization of Emotion Information Estimated from Neighboring User)

In addition, in the foregoing description, the case in which the emotion information is estimated from the biological information acquired by the wearable terminal 20 worn on the user has been described as the example. On the other hand, the information processing device 30 according to the embodiment may estimate the emotion information from the biological information acquired from another user around the user and generate the biology-emotion DB 390 in the instruction of the emotion information. At this time, the wearable terminal 20 worn on the other user and the information processing device 30 may be connected via the network 40.

### 3. Second Embodiment

Next, a second embodiment of the present disclosure will be described. In the above-described first embodiment, the case in which the operation of the operation device 10 which is an imaging device is controlled according to the emotion information of the user estimated by the information processing device 30 has been described as the example. On the other hand, the operation device 10 according to the embodiment may be, for example, a glasses type wearable terminal. The information processing device 30 according to the embodiment can perform display control related to the operation device 10 according to emotion information related to “interest” of the user estimated from biological information.

Note that in second to eleventh embodiments of the present disclosure to be described below, differences from the first embodiment will be mainly described and the repeated functions of the information processing device 30 will not be described. In addition, in the second to eleventh embodiments, a case in which the information processing device 30 includes the signal processing unit 320, the behavior recognition unit 330, the emotion estimation unit 340, the control unit 350, and the feedback recognition unit 380 will be described as an example. In addition, the operation device 10 may be a device that includes the behavior measurement sensor unit 315, the operation unit 360, and the input reception unit 370. In addition, the wearable terminal 20 may include the biological sensor unit 310.

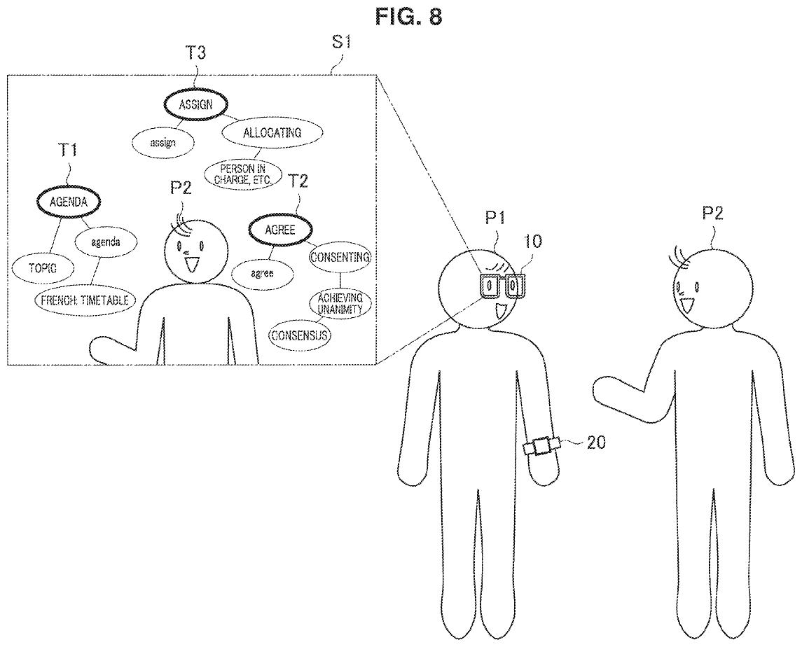

FIG. 8 is a conceptual diagram illustrating an overview according to the second embodiment of the present disclosure. Referring to FIG. 8, the operation device 10 according to the embodiment may be a glasses type wearable terminal which the user P1 wears. The information processing device 30 according to the embodiment may estimate emotion information related to “interest” of the user P1 from sound information in a conversation between the user P1 and another user P2 and may control an operation of the operation device 10 according to the emotion information

Specifically, the information processing device 30 according to the embodiment can retrieves a meaning or the like of a keyword extracted from previously spoken content and cause the operation device 10 to display the meaning or the like of the keyword in a case in which the emotion information related to “interest” of the user P1 is estimated from the sound information. In this case, the information processing device 30 may generate an interest-biology DB in advance by adding “interest” as a weak instruction to the biological information when the user retrieves the keyword.

In the example illustrated in FIG. 8, a display unit S1 of the operation device 10 controlled by the information processing device 30 is illustrated. In addition, on the display unit S1, pieces of text information T1 to T3 displayed by the operation device 10 according to control by the information processing device 30 are displayed. In this way, the information processing device 30 according to the embodiment can cause the operation device 10 to display text information or image information acquired on the Internet or the like according to “interest” of the user estimated in response to sound recognition. Note that a view range of the user P1 through the display unit S1 realized by see-through glass or the like is exemplified in FIG. 8.

In addition, the information processing device 30 according to the embodiment may correct correlation strength related to the interest-biology DB using a visual line of the user as feedback. The information processing device 30 can perform control such that the correlation strength is weakened, for example, according to the fact that the user does not orient his or her visual line to the pieces of text T1 to T3 displayed on the display unit S1.

### 4. Third Embodiment

Next, a third embodiment of the present disclosure will be described. The information processing device 30 according to the embodiment can perform operation control related to the operation device 10 according to emotion information related to “sleepiness” of the user estimated from biological information. Here, the emotion information related to the foregoing “sleepiness” may be associated with yawning of the user. The wearable terminal 20 can detect yawning of the user using a myoelectric sensor and transmit the detected information to the information processing device 30.

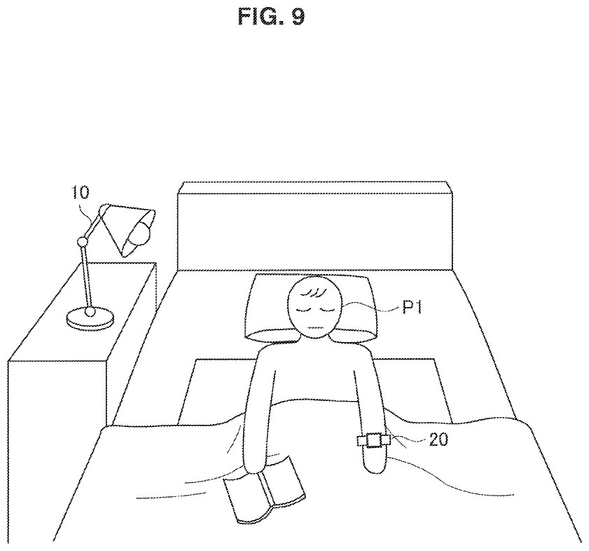

FIG. 9 is a conceptual diagram illustrating an overview according to the third embodiment of the present disclosure. Referring to FIG. 9, the operation device 10 according to the embodiment may be a lighting device that has a function of performing connection with the network 40. The information processing device 30 according to the embodiment can estimate the emotion information related to “sleepiness” of the user from biological information of the user acquired from the wearable terminal 20 and can perform lighting control related to the operation device 10 according to the emotion information.

In this case, the information processing device 30 may generate a sleepiness-biology DB in advance by adding “sleepiness” as a weak instruction to biological information such as a pulse wave, an electrocardiogram, or perspiration included for a predetermined time before and after the user yawns. According to the acquired biological information and “sleepiness” of the user estimated by the foregoing sleepiness-biology DB, the information processing device 30 according to the embodiment can perform control such that lighting of the operation device 10 is weakened or can perform such that luminescent color is changed into a warm color.

In addition, the information processing device 30 according to the embodiment may correct correlation strength related to the sleepiness-biology DB using a lighting operation of the user as feedback. The information processing device 30 can perform control such that the foregoing correlation strength is weakened, for example, according to the fact that the user strengthens the lighting of the operation device 10.

### 5. Fourth Embodiment

Next, a fourth embodiment of the present disclosure will be described. The information processing device 30 according to the embodiment can perform operation control related to the operation device 10 according to emotion information related to “boredom” of the user estimated from biological information. Here, the emotion information related to the foregoing “boredom” may be associated with a visual line of the user. The wearable terminal 20 can detect that the visual line of the user is not changed for a predetermined time or more and transmit the detected information to the information processing device 30. At this time, the wearable terminal 20 may determine the visual line of the user according to imaging information acquired by a visual line measurement sensor or the like.

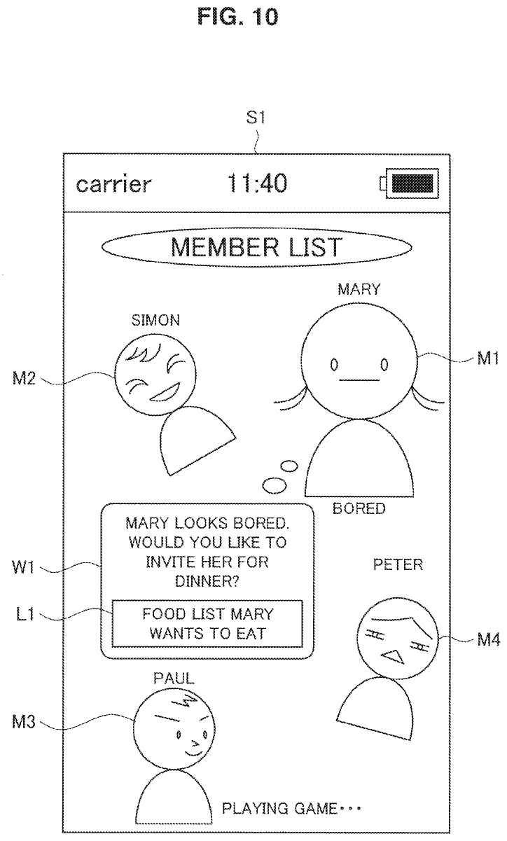

FIG. 10 is a conceptual diagram illustrating an overview according to the fourth embodiment of the present disclosure. Referring to FIG. 10, the operation device 10 according to the embodiment may be an information processing terminal such as a smartphone. The information processing device 30 according to the embodiment can estimate emotion information related to “boredom” of the user from biological information of the user acquired from the wearable terminal 20 and can perform control of an application related to the operation device 10 according to the emotion information. Specifically, the information processing device 30 according to the embodiment may change a user state in a chatting application to “bored” or the like. In addition, the information processing device 30 may perform control of display in the chatting application.

Referring to FIG. 10, a screen related to the chatting application is displayed on the display unit S1 of the operation device 10. In addition, icons representing members M1 to M4 are displayed on the screen. In the example illustrated in FIG. 10, the information processing device 30 changes the state of the member M1 to “bored” according to the emotion information related to “boredom” estimated from the biological information of the user corresponding to the member M1. In addition, the information processing device 30 causes a window W1 for the other members M2 to M4 to be displayed in association with the member M1. At this time, the information processing device 30 may cause a list L1 to be included in the window W1 and displayed. The information processing device 30 may control content to be caused to be displayed in the window W1 or the list L1 according to the acquired biological information of the user.

In this case, the information processing device 30 may generate a boredom-biology DB in advance by adding “boredom” as a weak instruction to biological information such as a pulse wave, an electrocardiogram, or perspiration included for a predetermined time before and after the detection according to the fact that the wearable terminal 20 detects that the visual line of the user has not changed for a predetermined time or more.

In addition, the information processing device 30 according to the embodiment may correct correlation strength related to the boredom-biology DB using a chatting application state manipulation by the user as feedback. The information processing device 30 can perform control such that the foregoing correlation strength is weakened, for example, according to the fact that the user changes the chatting application state into “busy” or the like.

### 6. Fifth Embodiment

Next, a fifth embodiment of the present disclosure will be described. The information processing device 30 according to the embodiment can perform operation control related to the operation device 10 according to emotion information related to “surprise” of the user estimated from biological information. Here, the emotion information related to the foregoing “surprise” may be associated with an instantaneous operation of the user. That is, in the embodiment, a motion of a body observed when the user is surprised may be recognized as behavior information. At this time, the wearable terminal 20 can detect the instantaneous operation of the user using an acceleration sensor and transmit the detected information to the information processing device 30.

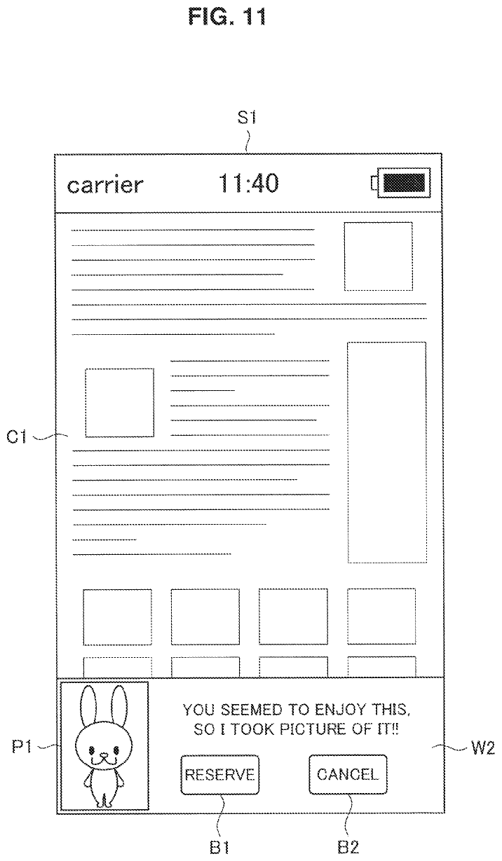

FIG. 11 is a conceptual diagram illustrating an overview according to the fifth embodiment of the present disclosure. Referring to FIG. 11, the operation device 10 according to the embodiment may be an information processing device such as a smartphone. The information processing device 30 according to the embodiment can estimate emotion information related to “surprise” of the user from biological information of the user acquired from the wearable terminal 20 and can perform display control related to the operation device 10 according to the emotion information. Specifically, the information processing device 30 according to the embodiment can cut a frame image related to a time at which “surprise” of the user is estimated from a moving image acquired by a sphere camera or the like and cause the operation device 10 to display the frame image.

Referring to FIG. 11, a screen related to content C1 and a window W2 are displayed on the display unit 51 of the operation device 10. In addition, an image P1, a button B1, and a button B2 are included in the window W2. Here, the image P1 displayed in the window W2 may be a frame image acquired according to a moving image acquired by a sphere camera or the like. In addition, the buttons B1 and B2 may be buttons used to perform a manipulation related to reservation or cancellation of each image.

The information processing device 30 according to the embodiment may generate a surprise-biology DB in advance by adding “surprise” as a weak instruction to the biological information such as a pulse wave, an electrocardiogram, or perspiration included for a predetermined time before and after the detection according to the fact that the wearable terminal 20 detects an instantaneous motion of the body of the user.

In addition, the information processing device 30 according to the embodiment may correct correlation strength related to the surprise-biology DB using a manipulation on the button B1 or B2 by the user as feedback. The information processing device 30 can perform control such that the foregoing correlation strength is weakened, for example, according to the fact that the user has pressed the button B2 related to the cancellation of the image P1. Further, the information processing device 30 can also perform control such that association related to the behavior-emotion DB 395 is weakened, for example, according to the fact that the user has continuously cancelled the image P1.

### 7. Sixth Embodiment

Next, a sixth embodiment of the present disclosure will be described. The information processing device 30 according to the embodiment can perform operation control related to the operation device 10 according to emotion information related to “sadness” of the user estimated from biological information. Here, the emotion information related to the foregoing “sadness” may be associated with tears of the user. The operation device 10 according to the embodiment can detect the tears of the user using a lacrimal gland sensor or the like and transmit the detected information to the information processing device 30. Therefore, the operation device 10 according to the embodiment may further include the biological sensor unit 310.

FIG. 12 is a conceptual diagram illustrating an overview according to the sixth embodiment of the present disclosure. Referring to FIG. 12, the operation device 10 according to the embodiment may be a contact wearable terminal. The information processing device 30 according to the embodiment can estimate the emotion information related to “sadness” of the user according to the tears of the user detected by the operation device 10 and cause the operation device 10 to display a proverb or the like according to the emotion information. In the example illustrated in FIG. 12, the information processing device 30 causes text information T4 related to the proverb to be displayed on the display unit S1 of the operation device 10. Note that, at this time, the information processing device 30 can estimate “sadness” of the user further according to sound recognition by a microphone or operation recognition by an acceleration sensor in addition the detection of the tears by the lacrimal gland sensor. In this case, the information processing device 30 can distinguish tears of the user which are not caused due to “sadness” and realize more highly precise emotion estimation.

In this case, the information processing device 30 may generate a sadness-biology DB in advance by adding “sadness” as a weak instruction to the biological information such as a pulse wave, an electrocardiogram, or perspiration included for a predetermined time before and after the user tears. The information processing device 30 according to the embodiment can retrieve a proverb or the like related to encouragement on the Internet or the like according to “sadness” of the user estimated from the foregoing sadness-biology DB and the acquired biological information and can cause the operation device 10 to display information related to the proverb.

In addition, the information processing device 30 according to the embodiment may correct correlation strength related to the sadness-biology DB using a visual line of the user as feedback. The information processing device 30 can perform control such that the foregoing correlation strength is weakened, for example, according to the fact that the user does not orient his or her visual line to text T4 displayed on the display unit S1.

### 8. Seventh Embodiment

Next, a seventh embodiment of the present disclosure will be described. The information processing device 30 according to the embodiment can perform operation control related to the operation device 10 according to emotion information related to “pleasure” of the user estimated from biological information. Here, the emotion information related to the foregoing “pleasure” may be associated with a smile of the user. The wearable terminal 20 can detect the smile of the user using a myoelectric sensor and transmit the detected information to the information processing device 30.

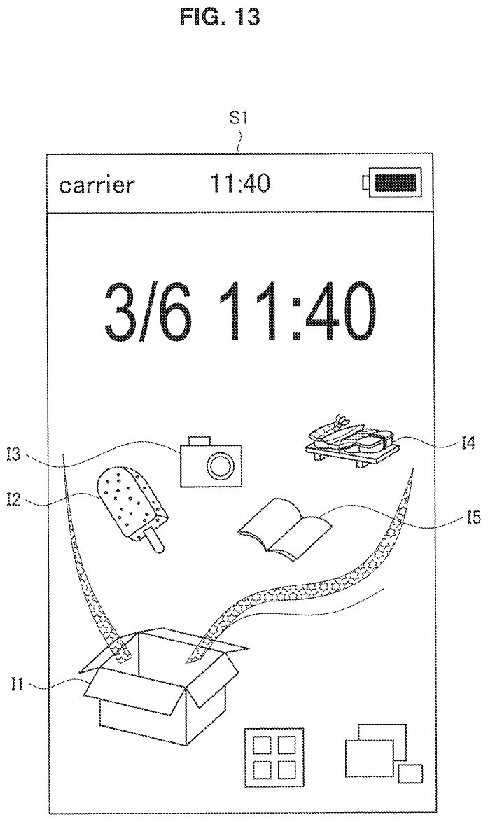

FIG. 13 is a conceptual diagram illustrating an overview according to the seventh embodiment of the present disclosure. Referring to FIG. 13, the operation device 10 according to the embodiment may be an information processing terminal such as a smartphone. The information processing device 30 according to the embodiment can estimate emotion information related to “pleasure” of the user from biological information of the user acquired from the wearable terminal 20 and can perform function control related to the operation device 10 according to the emotion information.

Specifically, the information processing device 30 according to the embodiment can cause the operation device 10 to recognize object in a surrounding environment of the user according to the estimation of the emotion information related to “pleasure” of the user and cause the operation device 10 to store the object as “lucky item.” At this time, the operation device 10 may perform object recognition using a scheme for object recognition, human recognition, or the like to be wisely used.

The information processing device 30 according to the embodiment may generate a pleasure-biology DB in advance by adding “pleasure” as a weak instruction to biological information such as a pulse wave, an electrocardiogram, or perspiration included for a predetermined time before and after the detection of the smile of the user.

In addition, the information processing device 30 according to the embodiment may control display of the operation device 10 related to the foregoing “lucky item.” Referring to FIG. 13, a home screen of an OS is displayed on the display unit S1 of the operation device 10. In addition, at this time, icons I1 to I4 related to “lucky item” are displayed on the foregoing home screen. Here, the icon I1 may be an application button or the like related to “lucky item” disposed on the home screen. In addition, the icons I2 to I4 may be image information related to “lucky item” developed when the item I1 is pressed. Note that the image information related to the icons I2 to I4 may be image information set according to the foregoing object recognition. In this way, the information processing device 30 according to the embodiment can control display of the operation device 10 related to “lucky item.”

In addition, the information processing device 30 according to the embodiment may correct correlation strength related to the pleasure-biology DB using a screen manipulation by the user as feedback. The information processing device 30 can perform control such that the foregoing correlation strength is weakened, for example, according to the fact that the user deletes “lucky item.”

### 9. Eighth Embodiment

Next, an eighth embodiment of the present disclosure will be described. The information processing device 30 according to the embodiment can perform operation control related to the operation device 10 according to emotion information related to “happiness” of the user estimated from biological information. Here, the emotion information related to the foregoing “happiness” may be associated with posting to a social networking service (SNS) by the user. The operation device 10 can detect the posting of the SNS by the user and transmit the detected information to the information processing device 30.

The information processing device 30 according to the embodiment may generate a happiness-biology DB in advance by adding “happiness” as a weak instruction to the biological information such as a pulse wave, an electrocardiogram, or perspiration included for a predetermined time before and after the detection of the SNS posting by the user.

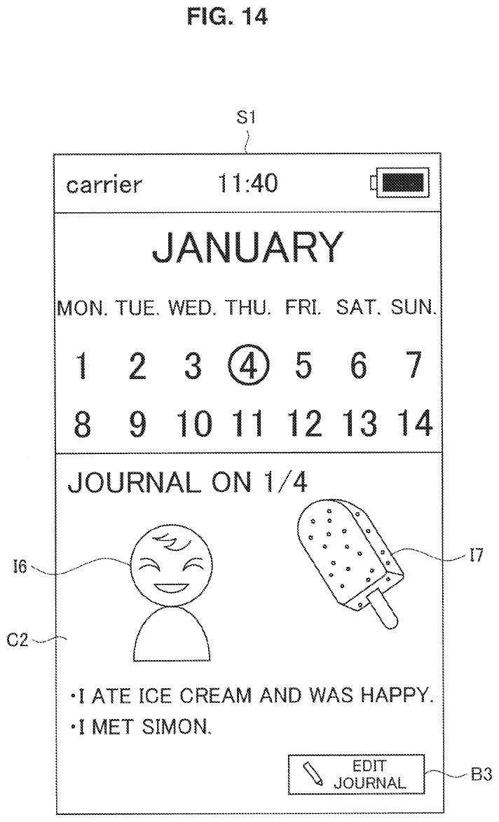

FIG. 14 is a conceptual diagram illustrating an overview according to the eighth embodiment of the present disclosure. Referring to FIG. 14, the operation device 10 according to the embodiment may be an information processing terminal such as a smartphone. The information processing device 30 according to the embodiment can estimate emotion information related to “happiness” of the user from biological information of the user acquired from the wearable terminal 20 and can perform function control related to the operation device 10 according to the emotion information.

Specifically, in a case in which the emotion information related to “happiness” of the user is estimated, the information processing device 30 according to the embodiment can cause the operation device 10 to recognize an object or a person in the surrounding environment of the user and can cause the operation device 10 to keep a journal based on a recognition result. At this time, the foregoing journal may include text information or image information automatically generated according to a result of the object recognition or the human recognition.

FIG. 14 illustrates an example of the journal displayed on the display unit S1 of the operation device 10. Referring to FIG. 14, the above-described text information or icons 16 and 17 based on the result of the object recognition or the human recognition are displayed in content C2 related to the journal. In addition, a button B3 for editing the journal is disposed in the content C2 related to the journal.

At this time, the information processing device 30 according to the embodiment may correct correlation strength related to the happiness-biology DB using a screen manipulation by the user as feedback. The information processing device 30 can perform control such that the foregoing correlation strength is weakened, for example, according to the fact that the user has pressed the button B3 and has edited the journal.

### 10. Ninth Embodiment

Next, a ninth embodiment of the present disclosure will be described. The information processing device 30 according to the embodiment can perform operation control related to the operation device 10 according to emotion information related to “unhappiness” of the user estimated from biological information. Here, the emotion information related to the foregoing “unhappiness” may be associated with a visual line of the user. The wearable terminal 20 can detect that the visual line of the user is oriented downward for a predetermined time or more and transmit the detected information to the information processing device 30. At this time, the wearable terminal 20 may determine the visual line of the user according to imaging information acquired by a visual line measurement sensor or the like.

In addition, according to the fact that the wearable terminal 20 detects that the visual line of the user has been oriented downward for a predetermined time, the information processing device 30 according to the embodiment may generate an unhappiness-biology DB in advance by adding “unhappiness” as a weak instruction to the biological information such as a pulse wave, an electrocardiogram, or perspiration included for a predetermined time before and after the detection.

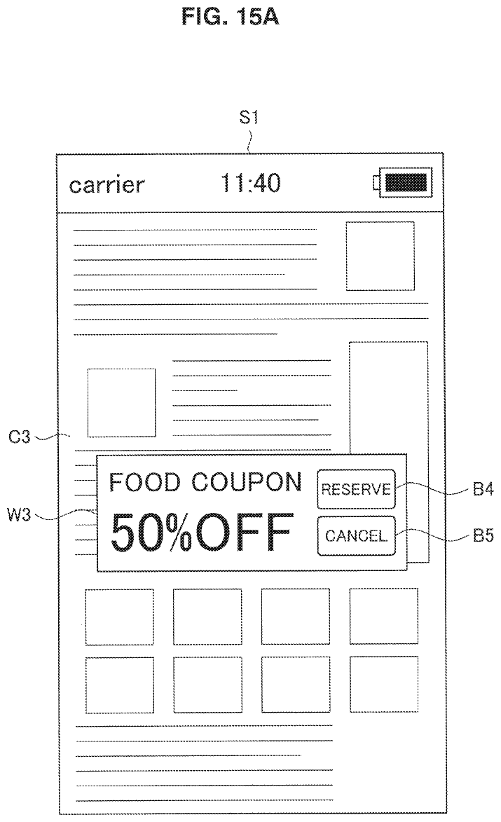

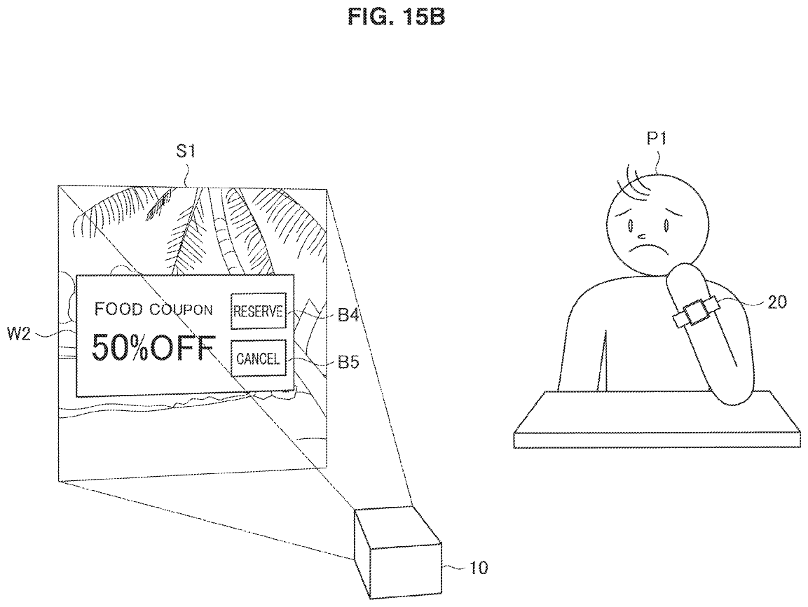

FIG. 15A is a conceptual diagram illustrating an overview according to the ninth embodiment of the present disclosure. Referring to FIG. 15A, the operation device 10 according to the embodiment may be an information processing terminal such as a smartphone. The information processing device 30 according to the embodiment can estimate emotion information related to “unhappiness” of the user from biological information of the user acquired from the wearable terminal 20 and can perform display control related to the operation device 10 according to the emotion information. Specifically, the information processing device 30 according to the embodiment can acquire an electronic coupon available in stores and cause the operation device 10 to display the electronic coupon.

Referring to FIG. 15A, a window W3 related to the display of the above-described electronic coupon on the content C3 browsed by the user is displayed on the display unit S1 of the operation device 10. In addition, buttons B4 and B5 related to reservation or cancellation of the electronic coupon are displayed in the window W3.

In addition, FIG. 15B is a diagram illustrating another control example of the operation device 10 to the embodiment. In the example illustrated in FIG. 15B, the operation device 10 may be a desk projector or the like. In this way, the information processing device 30 according to the embodiment may recognize the operation device 10 around the user via the network 40 or the like and perform display control of the recognized operation device 10.

In addition, the information processing device 30 according to the embodiment may correct correlation strength related to the unhappiness-biology DB using a button manipulation by the user as feedback. The information processing device 30 can perform control such that the foregoing correlation strength is weakened, for example, according to the fact that the user has pressed the button B5 related to the cancellation of the electronic coupon.

### 11. Tenth Embodiment

Next, a tenth embodiment of the present disclosure will be described. The information processing device 30 according to the embodiment can perform operation control related to the operation device 10 according to emotion information related to “concentration” of the user estimated from biological information. Here, the emotion information related to the foregoing “concentration” may be associated with an attitude of the user. The wearable terminal 20 can detect the attitude of the user when the user takes a seat using a gyro sensor and transmit the detected information to the information processing device 30.

In addition, according to the fact that the wearable terminal 20 has detects that the attitude of the user is good, the information processing device 30 according to the embodiment may generate a concentration-biology DB in advance by adding “concentration” as a weak instruction to the biological information such as a pulse wave, an electrocardiogram, or perspiration included for a predetermined time before and after the detection.

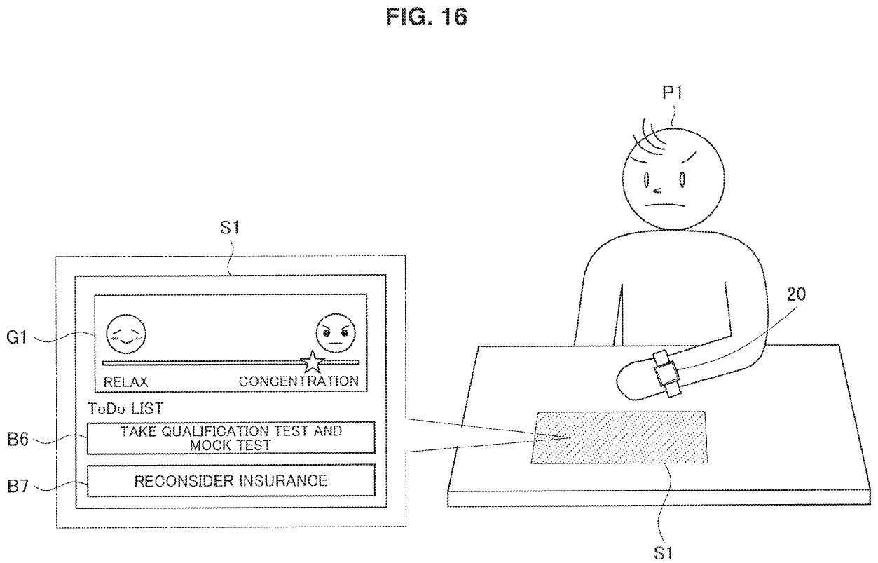

FIG. 16 is a conceptual diagram illustrating an overview according to the tenth embodiment of the present disclosure. Referring to FIG. 16, the operation device 10 according to the embodiment may be a desk display. The information processing device 30 according to the embodiment can estimate the emotion information related to “concentration” of the user from biological information of the user acquired from the wearable terminal 20 and can perform display control related to the operation device 10 according to the emotion information. Specifically, the information processing device 30 according to the embodiment may cause the operation device 10 to display an indicator, a ToDo list, or the like indicating the degree of concentration.

Referring to FIG. 16, the operation device 10 displays the indicator G1 representing the degree of concentration and ToDo lists B6 and B7 on the display unit S1. At this time, the user may select the ToDo list B6 or B7 to transition to each corresponding application screen.

In addition, the information processing device 30 according to the embodiment may correct correlation strength related to the concentration-biology DB using a screen manipulation by the user as feedback. The information processing device 30 can perform control such that the foregoing correlation strength is weakened, for example, according to the fact that the user has not selected the ToDo list B6 or B7 for a predetermined time or more.

### 12. Eleventh Embodiment

Next, an eleventh embodiment of the present disclosure will be described. The information processing device 30 according to the embodiment can perform operation control related to the operation device 10 according to emotion information related to “excitement” of the user estimated from biological information. Here, the emotion information related to the foregoing “excitement” may be associated with a motion of a hand line of the user. For example, in a case in which the user is watching a soccer game and a goal has been scored, the wearable terminal 20 can detect that the user has raised his or her hands using an acceleration sensor and transmit the detected information to the information processing device 30. In addition, the wearable terminal 20 may estimate that the user is watching a soccer game, for example, according to information acquired from GPS or time information.

In addition, according to the fact that the wearable terminal 20 detects that the user has raised his or her hands, the information processing device 30 according to the embodiment may generate an excitement-biology DB in advance by adding “excitement” as a weak instruction to the biological information such as a pulse wave, an electrocardiogram, or perspiration included for a predetermined time before and after the detection.

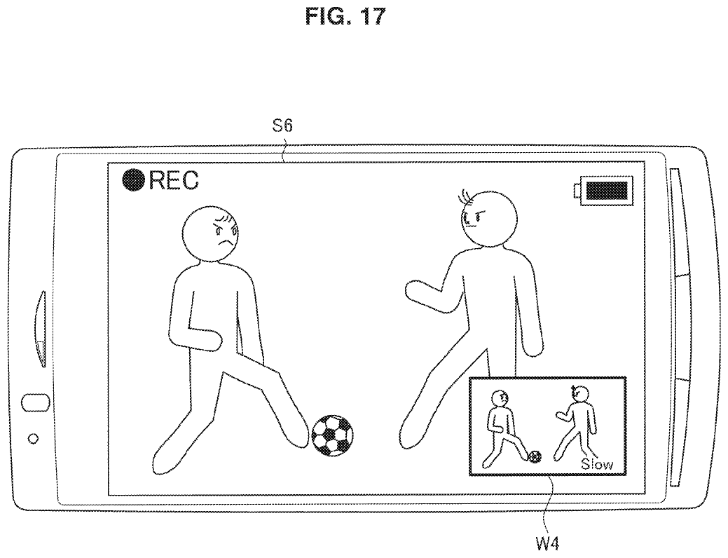

FIG. 17 is a conceptual diagram illustrating an overview according to the eleventh embodiment of the present disclosure. Referring to FIG. 17, the operation device 10 according to the embodiment may be a device that has a recording function such as a video camera or a smartphone. The information processing device 30 according to the embodiment can estimate emotion information related to “excitement” of the user from biological information of the user acquired from the wearable terminal 20 and can perform function control related to the operation device 10 according to the emotion information. Specifically, the information processing device 30 according to the embodiment can cause the operation device 10 to perform slow motion photographing and cause a photographed slow video to be displayed on the display unit S1 of the operation device 10.

Referring to FIG. 17, it can be understood that the slow video is displayed in a window W4 on the display unit S1 of the operation device 10 in addition to a video at the time of normal recording.

In addition, the information processing device 30 according to the embodiment may correct correlation strength related to the excitement-biology DB using a screen manipulation by the user as feedback. The information processing device 30 can perform control such that the foregoing correlation strength is weakened, for example, according to the fact that the user has switched the window W4 to non-display in the slow video.

### 13. Hardware Configuration Example

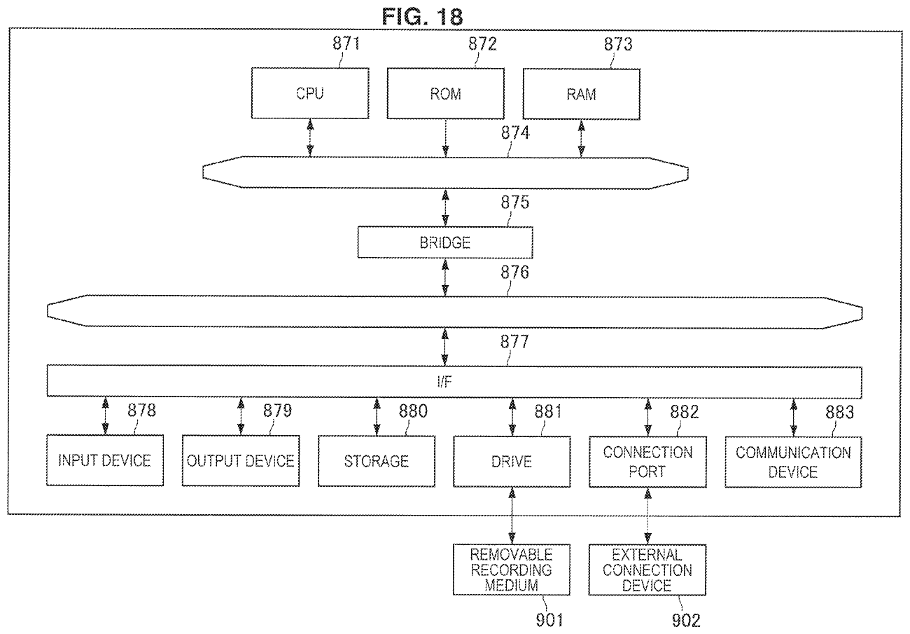

Next, a hardware configuration example common to the operation device 10, the wearable terminal 20, and the information processing device 30 according to the present disclosure will be described. FIG. 18 is a diagram illustrating a hardware configuration example of the operation device 10, the wearable terminal 20, and the information processing device 30 according to the present disclosure. Referring to FIG. 18, the operation device 10, the wearable terminal 20, and the information processing device 30 each include, for example, a CPU 871, ROM 872, a RAM 873, a host bus 874, a bridge 875, an external bus 876, an interface 877, an input device 878, an output device 879, a storage 880, a drive 881, a connection port 882, and a communication device 883. Note that the hardware configuration described here is exemplary and some of the constituent elements may be omitted. In addition, constituent elements other than the constituent elements described here may be further included.

### (CPU 871)

The CPU 871 functions as, for example, an arithmetic processing device or a control device and controls all or some of the operations of the constituent elements according to various programs recorded on the ROM 872, the RAM 873, the storage 880, or a removable recording medium 901.

### (ROM 872 and RAM 873)

The ROM 872 is means for storing data or the like to be used for calculation or a program to be read by the CPU 871. The RAM 873 temporarily or permanently a program to be read by the CPU 871, various parameters appropriately changed at the time of execution of the program, or the like.

### (Host Bus 874, Bridge 875, External Bus 876, and Interface 877)

For example, the CPU 871, the ROM 872, the RAM 873 are connected to each other via the host bus 874 capable of perform high-speed data transmission. On the other hand, the host bus 874 is connected to an external bus 876 of which a data transmission speed is relatively slow, for example, via the bridge 875. In addition, the external bus 876 is connected to various constituent elements via the interface 877.

### (Input Device 878)

For example, a mouse, a keyboard, a touch panel, a button, a switch, a lever, or the like is used as the input device 878. Further, a remote controller capable of transmitting a control signal using an infrared ray or other radio waves can also be used as the input device 878.

### (Output Device 879)

The output device 879 is, for example, a device capable of visually or audibly notifying a user of acquired information, such as a display device such as a cathode ray tube (CRT), an LCD, or an organic EL, an audio output device such as a speaker or a headphone, a printer, a mobile phone, or a facsimile.

### (Storage 880)

The storage 880 is a device that stores various kinds of data. For example, a magnetic storage device such as a hard disk drive (HDD), a semiconductor storage device, an optical storage device, or a magneto-optical storage device is used as the storage 880.

### (Drive 881)

The drive 881 is, for example, a device that reads information recorded on the removable recording medium 901 such as a magnetic disk, an optical disc, a magneto-optical disc, or a semiconductor memory or writes information on the removable recording medium 901.

### (Removable Recording Medium 901)

The removable recording medium 901 is, for example, a DVD medium, a Blu-ray (registered trademark) medium, an HD DVD medium, and any of various semiconductor storage media. Of course, the removable recording medium 901 may be, for example, an IC card on which a contactless IC chip or an electronic device.

### (Connection Port 882)

The connection port 882 is, for example, a port that connects the external connection device 902, such as a universal serial bus (USB) port, an IEEE1394 port, a small computer system interface (SCSI), an RS-232C port, or an optical audio terminal.

### (External Connection Device 902)

The external connection device 902 is, for example, a printer, a portable music player, a digital camera, a digital video camera, an IC recorder, or the like.

### (Communication Device 883)

The communication device 883 is a communication device that is connected to a network and is, for example, a wired or wireless LAN, a Bluetooth (registered trademark), a communication card for wireless USB (WUSB), a router for optical communication, a router for asymmetric digital subscriber line (ADSL), any of various communication modems, or the like.

### 14. Conclusion

As described above, the information processing device 30 according to the embodiment has the function of generating the biology-emotion DB 390 according to the behavior information and the biological information of the user. In addition, the information processing device 30 according to the embodiment has the function of controlling the operation of the operation unit 360 corresponding to the biological information of the user using the generated biology-emotion DB 390. In this configuration, it is possible to more efficiently collect data related to the emotions of the user and realize more highly precise emotion estimation.

The preferred embodiment(s) of the present disclosure has/have been described above with reference to the accompanying drawings, whilst the present disclosure is not limited to the above examples. A person skilled in the art may find various alterations and modifications within the scope of the appended claims, and it should be understood that they will naturally come under the technical scope of the present disclosure.

Further, the effects described in this specification are merely illustrative or exemplified effects, and are not limitative. That is, with or in the place of the above effects, the technology according to the present disclosure may achieve other effects that are clear to those skilled in the art from the description of this specification.

Additionally, the present technology may also be configured as below.

(1)

An information processing device including:

a control unit configured to acquire emotion information obtained from biological information of a user and output control information related to an operation of an operation unit corresponding to the biological information of the user according to behavior information associated with the emotion information.

(2)

The information processing device according to (1), further including:

a recognition unit configured to recognize feedback from the user on the operation of the operation unit,

in which the recognition unit controls correction related to correlation strength between the corresponding biological information and emotion information according to the feedback.

(3)

The information processing device according to (2), in which the recognition unit controls correction related to association between the corresponding behavior information and emotion information according to the feedback.

(4)

The information processing device according to (2) or (3), in which the feedback includes evaluation of the user in regard to the operation of the operation unit.

(5)

The information processing device according to (4), in which the recognition unit performs control such that the correlation strength between the corresponding biological information and emotion information is weakened according to negative evaluation of the user in regard to the operation of the operation unit.

(6)

The information processing device according to (4) or (5), in which the recognition unit performs control such that the correlation strength between the corresponding biological information and emotion information is strengthened according to positive evaluation of the user in regard to the operation of the operation unit.

(7)

The information processing device according to any of (4) to (6), in which the recognition unit performs control such that association between the corresponding behavior information and emotion information is deleted according to negative evaluation of the user in regard to the operation of the operation unit.

(8)

The information processing device according to any of (1) to (7),

in which the behavior information includes information indicating an operation that the operation unit is caused to perform, and

the control unit controls the operation that the operation unit is caused to perform according to the behavior information.

(9)

The information processing device according to any of (1) to (8), in which the emotion information includes a kind and degree of an emotion, and is acquired according to the biological information measured by a device carried by a user.

(10)

The information processing device according to (9),

in which the behavior information includes information related to a past device manipulation performed by a user, and

the control unit causes the operation unit to perform an operation similar to the device manipulation according to the behavior information associated with the kind and degree of the emotion.

(11)

The information processing device according to (9) or (10),

in which the behavior information includes information related to a device operation to be recommended to a user, and

the control unit causes the operation unit to perform the device operation to be recommended to the user according to the behavior information associated with the kind and degree of the emotion.

(12)

The information processing device according to (1), in which the control unit acquires the emotion information associated with the behavior information according to acquisition of behavior information of a user and causes a storage unit to store the emotion information and the acquired biological information in association with each other.

(13)

The information processing device according to (11), in which the device manipulation by the user is recognized by one of an acceleration sensor, a gyro sensor, a temperature sensor, a humidity sensor, a myoelectric sensor, a sound sensor, a pressure sensor, an imaging sensor, a microphone, a button, and a switch or a combination thereof.

(14)

An information processing method including, by a processor:

acquiring emotion information obtained from biological information of a user and outputting control information related to an operation of an operation unit corresponding to the biological information of the user according to behavior information associated with the emotion information.

(15)

A program causing a computer to function as an information processing device that includes a control unit configured to acquire emotion information obtained from biological information of a user and output control information related to an operation of an operation unit corresponding to the biological information of the user according to behavior information associated with the emotion information.

### REFERENCE SIGNS LIST

10 operation device
20 wearable terminal
30 information processing device
310 biological sensor unit
315 behavior measurement sensor unit 315
320 signal processing unit
330 behavior recognition unit
340 emotion estimation unit
350 control unit
360 operation unit
370 input reception unit
380 feedback recognition unit
390 biology-emotion DB
395 behavior-emotion DB

1. An information processing device comprising:
a control unit configured to acquire emotion information obtained from biological information of a user and output control information related to an operation of an operation unit corresponding to the biological information of the user according to behavior information associated with the emotion information.

2. The information processing device according to claim 1, further comprising:
a recognition unit configured to recognize feedback from the user on the operation of the operation unit,
wherein the recognition unit controls correction related to correlation strength between the corresponding biological information and emotion information according to the feedback.

3. The information processing device according to claim 2,
wherein the recognition unit controls correction related to association between the corresponding behavior information and emotion information according to the feedback.

4. The information processing device according to claim 2,
wherein the feedback includes evaluation of the user in regard to the operation of the operation unit.

5. The information processing device according to claim 4,
wherein the recognition unit performs control such that the correlation strength between the corresponding biological information and emotion information is weakened according to negative evaluation of the user in regard to the operation of the operation unit.

6. The information processing device according to claim 4, wherein the recognition unit performs control such that the correlation strength between the corresponding biological information and emotion information is strengthened according to positive evaluation of the user in regard to the operation of the operation unit.

7. The information processing device according to claim 4,
wherein the recognition unit performs control such that association between the corresponding behavior information and emotion information is deleted according to negative evaluation of the user in regard to the operation of the operation unit.

8. The information processing device according to claim 1,
wherein the behavior information includes information indicating an operation that the operation unit is caused to perform, and
the control unit controls the operation that the operation unit is caused to perform according to the behavior information.

9. The information processing device according to claim 1,
wherein the emotion information includes a kind and degree of an emotion, and is acquired according to the biological information measured by a device carried by a user.

10. The information processing device according to claim 9,
wherein the behavior information includes information related to a past device manipulation performed by a user, and
the control unit causes the operation unit to perform an operation similar to the device manipulation according to the behavior information associated with the kind and degree of the emotion.

11. The information processing device according to claim 9,
wherein the behavior information includes information related to a device operation to be recommended to a user, and
the control unit causes the operation unit to perform the device operation to be recommended to the user according to the behavior information associated with the kind and degree of the emotion.

12. The information processing device according to claim 1,
wherein the control unit acquires the emotion information associated with the behavior information according to acquisition of behavior information of a user and causes a storage unit to store the emotion information and the acquired biological information in association with each other.

13. The information processing device according to claim 11,
wherein the device manipulation by the user is recognized by one of an acceleration sensor, a gyro sensor, a temperature sensor, a humidity sensor, a myoelectric sensor, a sound sensor, a pressure sensor, an imaging sensor, a microphone, a button, and a switch or a combination thereof.

14. An information processing method comprising, by a processor:
acquiring emotion information obtained from biological information of a user and outputting control information related to an operation of an operation unit corresponding to the biological information of the user according to behavior information associated with the emotion information.

15. A program causing a computer to function as an information processing device that includes a control unit configured to acquire emotion information obtained from biological information of a user and output control information related to an operation of an operation unit corresponding to the biological information of the user according to behavior information associated with the emotion information.
